(19) United States
(12) Patent Application Publication
(10) Pub. No.: US 2008/0300546 A1
(43) Pub. Date: Dec. 4, 2008

(54) DEVICES AND METHODS FOR STABILIZING MEDICAL INSTRUMENTS

(75) Inventors: Neil Godara, Milton (CA); Erin Gibson, Guelph (CA); Taylor Hillier, Georgetown (CA)

Correspondence Address: DIMOCK STRATTON LLP, 20 QUEEN STREET WEST SUITE 3202, BOX 102, TORONTO, ON, M5H 3R3, CA

(73) Assignee: Baylis Medical Company Inc., Montreal (CA)

(21) Appl. No.: 11/689,902

(22) Filed: Mar. 22, 2007

Related U.S. Application Data
(60) Provisional application No. 60/743,664, filed on Mar. 22, 2006.

(51) Int. Cl. A61M 5/32

(52) U.S. Cl. 604/174

(57) ABSTRACT

Embodiments of a device, and method of use thereof, are described for stabilizing a medical instrument, the medical instrument contacting a patient's body at a point of contact, the method using a plurality of support members for stabilizing the medical instrument, each support member comprising an instrument end and a patient end and an elongate support member body extending therebetween, the method comprising: independently coupling the instrument end of each of the plurality of support members to the medical instrument; and independently attaching the patient end of each of the plurality of support members to a surface of the patient's body for stabilizing the medical instrument.

### REFERENCES TO PARENT AND CO-PENDING APPLICATIONS

This application claims the benefit of U.S. provisional application Ser. No. 60/743,664, filed Mar. 22, 2006, which is incorporated herein by reference.

### TECHNICAL FIELD

The invention relates to devices and methods for supporting or stabilizing medical instruments in or on a patient's body.

### BACKGROUND OF THE ART

Several medical procedures exist wherein a medical instrument must be held upright or at a particular angle in or on a patient's body. Often, a surgeon, nurse, or other user must manually hold such an instrument in place. This may be tedious or cumbersome for the user.

Examples of prior art structures utilized to maintain a medical instrument in an operating position with respect to tissue through which the instrument extends are shown in each of U.S. Pat. No. 4,579,120, issued on Apr. 1, 1986 to Macregor; U.S. Pat. No. 5,073,169, issued on Dec. 17, 1991 to Raiken; U.S. Pat. No. 5,201,742, issued on Apr. 13, 1993 to Hasson; U.S. Pat. No. 5,352,211, issued to Merskelly on Oct. 4, 1994; and U.S. Pat. No. 5,897,531, issued to Amirana on Apr. 27, 1999. In each of the above patents, a disk or similar supporting base with a large surface area is borne against one side of a tissue through which the instrument extends to thereby positively maintain the position of the instrument. The large surface area of the supporting base makes it difficult to position a plurality of instruments in proximity to one another, since they would necessarily have to be spaced apart by a distance equivalent to at least the radius of the supporting base. In addition, having a relatively large supporting base makes it difficult to reposition and/or re-orient the base if required during a treatment procedure.

### BRIEF DESCRIPTION OF THE DRAWINGS

In order that the invention may be readily understood, embodiments of the invention are illustrated by way of examples in the accompanying drawings, in which:

FIG. 1 is a perspective view of one embodiment of the present invention;

FIGS. 2A-B are top views of different versions of the instrument end of the support member of one embodiment of the present invention;

FIGS. 3A-B are perspective views of different versions of the patient end of the support member of one embodiment of the present invention;

FIG. 4 is a perspective view of an alternate embodiment of the present invention; and

FIG. 5 is a perspective view of another alternate embodiment of the present invention.

### DETAILED DESCRIPTION OF THE INVENTION

With specific reference now to the drawings in detail, it is stressed that the particulars shown are by way of example and for purposes of illustrative discussion of certain embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In one broad aspect, embodiments of the present invention comprise a method for stabilizing a substantially rigid medical instrument, the medical instrument contacting a patient's body at a point of contact, the method using a plurality of support members for stabilizing the medical instrument, each support member comprising an instrument end and a patient end and an elongate support member body extending therebetween, the method comprising: independently coupling the instrument end of each of the plurality of support members to the medical instrument; and independently attaching the patient end of each of the plurality of support members to a surface of the patient's body for stabilizing the medical instrument.

In another broad aspect, embodiments of the present invention comprise a method for stabilizing a plurality of substantially rigid medical instruments, the plurality of medical instruments contacting a patient's body in close proximity to one another, the method using a plurality of support members for stabilizing each of the plurality of medical instruments, each support member comprising an instrument end and a patient end and an elongate support member body extending therebetween, the support member body sized to position the patient end at a distance from a point of contact of the medical instrument with the patient's body, the method comprising: for each of the plurality of medical instruments, independently coupling the instrument end of each of the plurality of support members to the medical instrument; and, for each of the plurality of medical instruments, independently attaching the patient end of each of the plurality of support members to a surface of the patient's body, at a distance from the point of contact, for stabilizing the medical instrument; whereby attaching the patient end of each of the plurality of support members to a surface of the patient's body at a distance from the point of contact allows for overlapping a support member body associated with one of the plurality of medical instruments with at least a portion of a support member associated with another of the plurality of medical instruments, whereby the plurality of medical instruments may be stabilized in close proximity to one another.

In yet another broad aspect, the present invention provides, in various embodiments, a kit of parts for stabilizing a substantially rigid elongate medical instrument contacting a patient's body at a point of contact, the kit comprising: at least one instrument support member comprising an instrument end for removably coupling to the medical instrument, the instrument end at least partially defining an aperture sized for selectively movably affixing the instrument end to the medical instrument by frictional engagement, an elongate support member body extending from the instrument end for stabilizing the instrument end, and a patient end extending from the elongate support member body for removably attaching to the patient's body for anchoring the support member body; and at least one motion limiting member for limiting longitudinal motion of one or more of the medical instrument and the instrument end of the support member.

In an additional broad aspect, the present invention provides an apparatus, in various embodiments, for stabilizing a substantially rigid elongate medical instrument contacting a patient's body, the apparatus comprising: a plurality of independent instrument support members, each support member comprising an instrument end for removably coupling to the medical instrument, the instrument end adapted to be selectively movably affixed by frictional engagement to the medical instrument, an elongate support member body extending from the instrument end for stabilizing the instrument end, and a patient end extending from the elongate support member body for removably attaching to the patient's body for anchoring the support member body.

In a further broad aspect, embodiments of the present invention provide a device for stabilizing a substantially rigid medical instrument contacting a patient's body at a point of contact, the device comprising: an instrument end for removably coupling to the medical instrument, the instrument end at least partially defining an aperture sized for selectively movably affixing the instrument end to the medical instrument by frictional engagement; a plurality of elongate support member bodies extending from the instrument end for stabilizing the instrument end; and a patient end extending from each of the plurality of elongate support member bodies for removably attaching to the patient's body for anchoring the plurality of support member bodies.

Thus, embodiments of the present invention, as described herein below, provide devices, and methods of use thereof, useful for stabilizing medical instruments contacting or inserted within a patient's body. Embodiments of the devices and apparatuses described herein may be manufactured at a relatively low cost and may be selectively attachable and/or removable from the medical instrument. More particularly, embodiments of the present invention may be coupled to the medical instrument and/or may be attached to the patient's body after the medical instrument has been properly positioned at or within the patient's body. Furthermore, as described herein below, a plurality of medical instruments may be positioned in close proximity to one another while using embodiments of devices of the present invention to stabilize each of the medical instruments, since embodiments of devices of the present invention allow for stabilization of each medical instrument while allowing for positioning of the devices so as to avoid obstructing a neighboring instrument. In addition, in applications involving the insertion of medical instruments into the patient's body, embodiments of devices of the present invention described herein do not limit accessibility to the site at which the instruments are inserted.

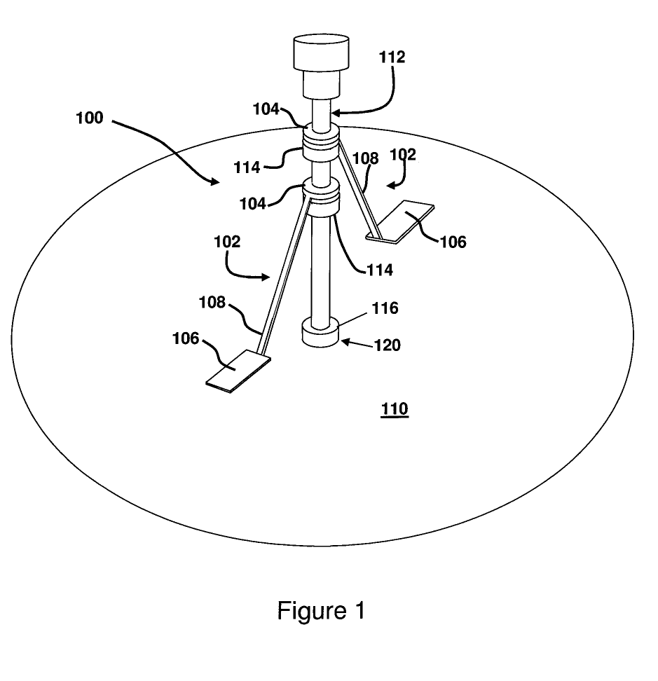

With reference now to the attached drawings, FIG. 1 shows an embodiment of an apparatus 100 of the present invention which, when operatively connected to a medical instrument 112, helps to stabilize the medical instrument 112. The medical instrument 112 may be any instrument that may require support, anchoring, or securing in or on the body of a patient. Examples of such medical instruments may include, but are not limited to, cannulae, trocars, needles, sheaths, probes, obturators, or stylets. As used herein, the term “cannula” refers to a substantially rigid elongate device defining a lumen while the term “obturator” refers to any item that substantially fills or blocks a lumen, for example a stylet.

In some embodiments, the medical instrument 112 may be a substantially rigid elongate element, such as a needle, probe, or cannula. Such instruments may tend to tip over or otherwise move during the course of a surgical procedure and may therefore benefit from a supporting device as disclosed herein. In embodiments used with a substantially rigid medical instrument, the medical instrument itself may, for example, form one leg of a tripod, as shown in FIG. 1. The other legs, referred to herein as ‘support members’ 102 may be any structure that may support or stabilize the medical instrument in a position determined by the user. The support members 102 may serve to limit transverse, longitudinal or angular movement of the medical instrument. Although two support members 102 are shown in FIG. 1, some embodiments, depending on the particularities of the medical instrument, the treatment procedure and/or the preferences of the user, may be practiced with an apparatus having any number of support members 102.

In one particular embodiment, as shown in FIG. 1, the support members 102 comprise elongate pieces of material that are operatively connected to the medical instrument 112 at one end, and to a surface of the patient's body, for example the patient's skin 110, at the other end. As used herein, the term “at”, for example when referring to something being located “at” a specific location, is intended to include any one or more of: proximate, on, near, adjacent to or within the specific location. Also, as used herein, the portion of the support member that is coupled to the medical instrument may be referred to as an ‘instrument end’ 104, the portion of the support member that is attached to the patient's body may be referred to as a ‘patient end’ 106, and the portion therebetween may be referred to as a ‘support member body’ 108. As will be described herein below, some embodiments of an apparatus of the present invention function to support medical instrument 112 substantially non-invasively, i.e. without requiring supplemental anchoring or support underneath the surface of the patient's body.

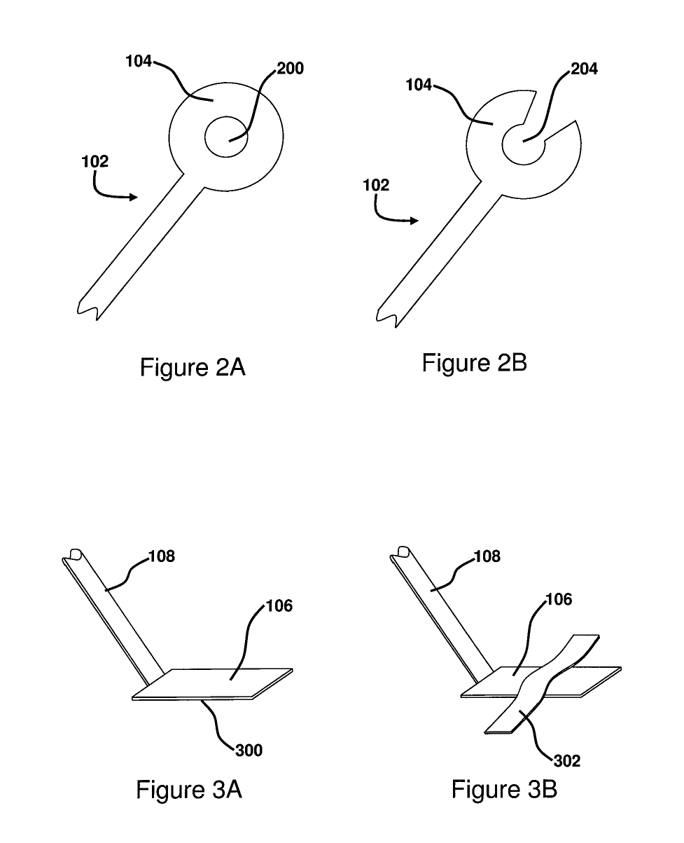

Support members 102 may be removably coupled to medical instrument 112 by a variety of means. For example, in one embodiment, instrument end 104 of support member 102 may define an aperture 200, sized to allow medical instrument 112 to pass therethrough, as shown in FIG. 2A. In another embodiment, instrument end 104 of support member 102 may partially define an aperture 204 such that instrument end 104 doesn't fully circumscribe aperture 204, as shown in FIG. 2B. In such an embodiment, medical instrument 112 may be snapped, pushed, or pressed into aperture 204. The aperture may be sized such that medical instrument 112 may fit tightly therein, such that medical instrument 112 may resist sliding within aperture 202 or aperture 204 unless a sufficient force is applied by a user. Thus, these embodiments provide an instrument end 104 for removably coupling to the medical instrument 112, the instrument end 104 at least partially defining an aperture sized for selectively movably affixing the instrument end 104 to the medical instrument 112 by frictional engagement.

Patient end 106 of support member 102 may be removably attached to the patient's skin 110 by a variety of means. In one embodiment, a patient-contacting surface of patient end 106 of support member 102 may comprise an adhesive 300, such that it may adhere to the patient's skin 110, as shown in FIG. 3A. Adhesive 300 may be, for example, any biocompatible or medical grade adhesive, such as a silicone adhesive or an acrylic co-polymer. Adhesive 300 may be in the form of a coating, for example, or may be in the form of a double sided tape, wherein one side adheres to patient end 106, and the other side adheres to the patient's skin 110. In another embodiment, as shown in FIG. 3B, patient end 106 of support member 102 may be attached to the patient's skin 110 by placing a surface of patient end 106 of support member 102 on the patient's skin 110, and using surgical tape 302 or other securing means to hold patient end 106 to the skin 110. In some minimally-invasive embodiments, needles, hooks or other securing means are inserted through patient end 106 and the patient's skin 110 in order to hold patient end 106 in position. Alternatively, patient end 106 may be removably attached to the patient's skin 110 by over-molding a patient contacting surface of patient end 106 with a friction member such as silicone. Such over-molding helps to secure the patient end 106 to the patient's skin 110 and may be particularly useful in applications where the patient's skin 110 is somewhat wet such that it may not be possible to attach patient end 106 to the patient's skin 110 using an adhesive. Thus, there are a number of securing means for securing patient end 106 of support member 110 to the skin of the patient, and the invention is not limited in this regard.

Thus, in some embodiments of the present invention, each support member 102 may be independently coupled to the instrument 112 at the instrument end 104 and, alternatively or in addition, may be independently attached to the patient's body at patient end 106. In the context of the present invention, ‘independently coupled’ means that each individual support member 102 may be coupled, removed and/or repositioned with respect to the instrument 112 independently of any other support members 102. Similarly, in the context of the present invention, ‘independently attached’ means that each individual support member 102 may be attached, removed and/or repositioned with respect to the patient's body independently of any other support members 102.

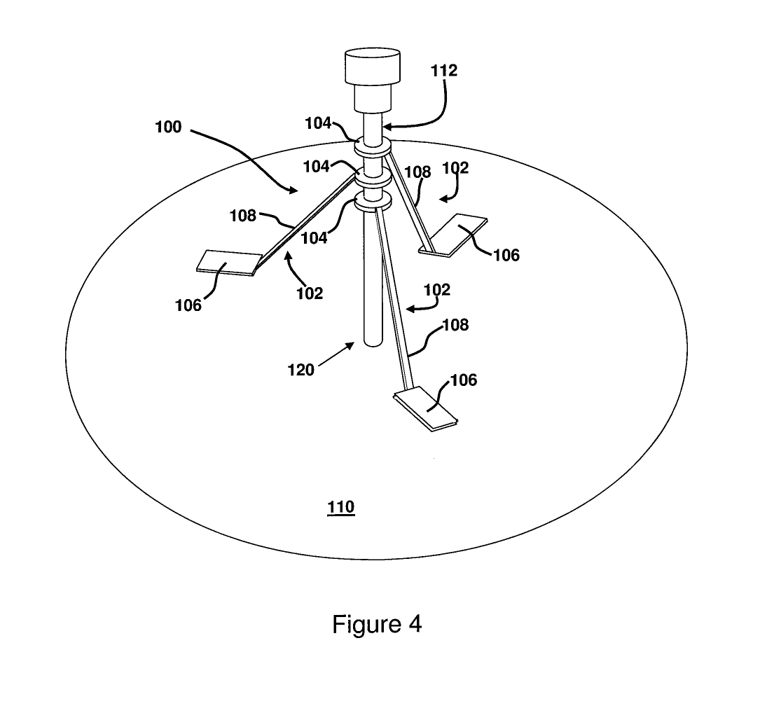

With reference now to FIG. 4, an alternate embodiment of a device of the present invention is provided. In this embodiment, rather than utilizing a plurality of support members 102, each having an instrument end 104, a member support body 108 and a patient end 106, a single support member 402 is used to stabilize medical instrument 112. Support member 402 comprises a single instrument end 104 for removably coupling to the medical instrument 112, a plurality of elongate support member bodies 108 extending from the instrument end 104 for stabilizing the instrument end 104, and at least one patient end 106 extending from each of the plurality of elongate support member bodies 108 for removably attaching to the patient's body for anchoring the plurality of support member bodies 108. In the illustrated embodiment, as described herein above, the instrument end 104 at least partially defines an aperture sized for selectively movably affixing the instrument end 104 to the medical instrument 112 by frictional engagement. Also, each patient end 106 may be attached to the patient's body independent of any other patient end 106.

In any of the aforementioned embodiments, support member body 108 may vary in size depending on the medical instrument to be held. For example, if medical instrument 112 is a 16 to 20 gauge cannula, more specifically a 17 gauge cannula, having a length of about 70 to about 80 mm, the support member body 108 may be between about 30 and 50 mm in length. It is to be noted, however, that the length of the support member body 108 depends on a number of other factors such as the intended use of the medical instrument and the angle at which the medical instrument is to be held (in general, if instrument end 104 is located further away from the patient's skin 110, then patient end 106 may be located closer to the point of contact 120, i.e. the point at which medical instrument 112 contacts the patient's body, if the length of support member body 108 remains constant), and the invention is therefore not limited in this regard. In some particular embodiments, the length of support member body 108 may be variable, such that support member body 108 is extendible and/or retractable and may be adjusted before or during the course of a treatment procedure.

In embodiments of the present invention, support member body 108 is elongate and is sized such that one or more of patient end 106 and instrument end 104 may be attached to the patient's body or to the medical instrument 112, respectively, at a distance from the point 120 at which medical instrument 112 contacts the patient's body 110. As described further herein below, attaching patient end 106 at a distance from this point of contact 120 allows for substantially unimpeded access to the point of contact 120 and may also allow for multiple medical instruments 112 to be positioned near each other, as described further herein below. In addition, attaching instrument end 104 to medical instrument 112 at a distance from the point of contact 120 (i.e. nearer the proximal end of medical instrument 112, where the proximal end refers to the end closer to the user when the device is in use) helps to reduce, or substantially limit, the angular motion of medical instrument 112 about the point of contact 120.

The width and depth of support member body 108 may be sized depending on the strength and flexibility required of support member body 108. For example, if the medical instrument 112 is a stainless steel 17 gauge cannula having a length of about 75 mm, a support member body 108 of about 3 mm in width and about 1 mm in depth may be sufficiently strong to hold the cannula in place. For a heavier and/or larger medical instrument, a larger width and depth may be required. Furthermore, the support member body 108 may be required to bend depending on the positioning and angle of the medical instrument with respect to the body, and therefore support member body 108 may be further sized such that it remains substantially flexible. In alternate embodiments, support member body 108 may be substantially rigid. The dimensions of support member body 108 may vary over a wide range without interfering with the functioning of the device, and the invention is therefore not limited in this regard.

Support member 102 may be manufactured from a number of different materials. Examples of suitable materials include, but are not limited to, medical grade plastics such as polypropylene, polycarbonate, polytetrafluoroethylene (PTFE) and silicone rubbers. In some embodiments, the material may be sterilizable. Furthermore, each of the aforementioned portions of the support member may be made from different materials. For example, a substantially stiff material may be desirable for instrument end 104 of support member 102, such that it may securely couple to medical instrument 112; whereas a more flexible material may be desired for support member body 108, such that it may bend to accommodate the position required by the user.

In some embodiments, and as shown in FIG. 1, the device may further comprise one or more motion limiting members for limiting motion, for example longitudinal motion, of one or more of medical instrument 112 and instrument end 104. In some embodiments, the means for preventing motion comprises a substantially annular component, for example a depth stopper 114, which may be disposed around, and slidably frictionally engaged with, medical instrument 112. In the context of the present invention, substantially annular includes components that are operable to surround a sufficient circumference of the medical instrument 112 in order to remain coupled to the medical instrument 112 including, but not limited to, penannular components. Depth stopper 114 may fit tightly around medical instrument 112, such that it may resist sliding or otherwise moving along medical instrument 112 due to gravity or small amounts of force, but may be repositioned if the user applies sufficient force. Depth stopper 114 may be manufactured from rubber or another resilient material. Depth stopper 114 may function to support instrument end 104 of support member 102 on medical instrument 112, in order to prevent support member 102 from sliding along the instrument.

Furthermore, as mentioned herein above, by positioning the instrument ends 104 of the support members 102 at a greater distance from the surface 110 of the patient's body, lower forces are required to relieve the moment (with the pivot point at the point of contact 120) caused by the weight of the medical instrument 112 and/or any cables, wires or other devices coupled to the medical instrument 112. In other words, the angular motion of medical instrument 112 about the point of contact 120 may be substantially reduced by positioning instrument end 104 further away from the point of contact 120. Depth stopper 114 may assist in positioning instrument end 104 at a desired location along medical instrument 112 by providing support to instrument end 104, thus ensuring that instrument end 104 doesn't slide down the shaft of medical instrument 112.

In some embodiments, the device may comprise multiple depth stoppers 114, 116. For example, in the embodiment shown in FIG. 1, a first depth stopper 114 may be used to support instrument end 104 of support member 102 while a second depth stopper 116 may be used to provide further stabilizing support for medical instrument 112 substantially adjacent point of contact 120. This second depth stopper 116 may also be used to prevent longitudinal motion of medical instrument 112 so as to ensure that medical instrument 112 is inserted to the patient's body at a desired depth. Thus, depth stoppers 114, 116 may be used to provide stabilizing support for the medical instrument 112 and to substantially prevent or limit longitudinal motion of the medical instrument 112 and/or the support members 102. In other words, the depth stopper(s) help to (1) limit or prevent longitudinal motion of the medical instrument and also to (2) offset the support ends, thus reducing the forces necessary to counter the moment created about the point of contact 120.

In some embodiments, a depth stopper 116 may further comprise a treatment composition including, but not limited to, a topical anesthetic or steroids, for example, which may be delivered to patient's body by placing depth stopper 116 adjacent the surface of the patient's body. In addition, in some minimally-invasive embodiments, depth stopper 116 may comprise a means for anchoring depth stopper 116 to the patient's body. The means for anchoring may comprise one or more projections for piercing the skin 110 of the patient's body to further stabilize medical instrument 112.

In further embodiments of the present invention, the device may comprise one or more markings, for example visual, tactile or radiopaque markings, for assisting in visualization under fluoroscopy. In addition, one or more components of the device may be substantially radiolucent or radiopaque. For example, depth stopper 116 may be radiolucent or substantially radiopaque such that, when depth stopper 116 is positioned at the point of contact 120, the point of contact 120 may be more readily visualized using fluoroscopic imaging. Alternatively, or in addition, depth stopper 116 may be fabricated from a substantially luminous material for providing improved visualization of the point of contact 120. This may be particularly beneficial when multiple medical instruments 112 are positioned within a relatively small area on the surface 110 of the patient's body. Furthermore, such embodiments may be useful for aligning the supporting device with a portion of a medical instrument located within the patient's body.

In some embodiments, one or more components of an apparatus 100 may be color-coded for easier identification. This color-coding may depend, for example, on the size of the component. For example, if support members 102 and/or depth stoppers 114, 116 are sized to be used with a particular medical instrument 112, for example depending on the gauge of the medical instrument 112, then one or more of support members 102 and depth stoppers 114, 116 may be color-coded (or otherwise identifiable) so as to be readily associated with such a particularly-sized medical instrument 112.

### METHOD

In the context of the present invention, a medical instrument ‘contacting’ the patient's body may be positioned with its distal portion at the surface of the patient's body or the distal portion may be inserted through the surface to a desired location within the patient's body. The location at which the medical instrument contacts the patient's body, whether or not it is inserted through the surface of the patient's body, is referred to as the point of contact as described herein above.

In one embodiment, the method of the present invention may be used during a surgical procedure that may require an instrument such as a needle, probe, or cannula to be inserted into the body of a patient and held at a particular angle or position for a certain amount of time. Such procedures may include, for example, minimally invasive electrosurgical procedures, in which a medical instrument is advanced into the body to a target site, such as a nerve, and energy is delivered from the medical instrument to the target site while the medical instrument is held in place. One example of such a procedure is radiofrequency ablation of a target nerve in the sacroiliac region of a patient's body.

Some embodiments of method aspects of the present invention may, in addition to securing or stabilizing a medical instrument, further include additional steps, including but not limited to: preparing a patient for a treatment procedure, inserting a medical instrument into a target site within the patient's body and treating the target site using the medical instrument.

In one embodiment of a patient preparation step, a user may utilize diagnostic techniques to identify a target site within the body. As mentioned hereinabove, such a target site may be a nerve or group of nerves within the sacroiliac region of a patient's body, for example. In some embodiments, a marker may be placed on the patient's body to indicate proper placement of a medical instrument in order to treat the target site. In some such embodiments, this marker may take the form of a depth stopper as described herein above for stabilizing the medical instrument. In some embodiments, the patient may be prepared for the treatment procedure by administering anesthetics, sedatives, or any other suitable compounds or pharmaceuticals. Furthermore, any entry sites into the body may be cleaned, disinfected, or otherwise prepared.

Regarding the step of inserting a medical instrument into a target site, and using a sacroiliac pain treatment procedure as an example, the user may advance a medical instrument, for example an obturator disposed within a cannula, towards the target site within the sacroiliac region. The depth of advancement may depend on several factors, such as the size and weight of the patient, and the specific anatomical structure of the patient's sacroiliac region. In general, a certain length of cannula may remain outside of the patient's body when the medical instrument has reached the target site. The user may then withdraw the obturator from the cannula, and insert an electrosurgical device, such as a probe comprising an energy delivery means, into the cannula. When the energy delivery means is properly placed within the body, energy may be delivered from the energy delivery means to the target site to treat the target site.

In embodiments of the method aspect of the present invention, the user may desire to maintain the position of a medical instrument such as those described hereinabove for a period of time, for example until energy delivery has been completed. In order to maintain the position of the medical instrument, the user may utilize an apparatus of the present invention to secure and/or stabilize the medical instrument, such that the medical instrument remains substantially static during the course of a treatment procedure. For example, the apparatus shown in FIG. 1 and/or its equivalents may be used to support the medical instrument in a desired position.

In some embodiments, the user may couple an end of a supporting device, such as instrument end 104 of support member 102, described herein above, to a medical instrument, such as a cannula. Such a coupling may be facilitated by snapping or pushing the portion of the medical instrument that remains outside of the body into an aperture or divot, for example aperture 204, defined by instrument end 104 of support member 102. Alternatively, prior to inserting the medical instrument into the patient's body, the user may pass the medical instrument through an aperture in the support member, such as aperture 200 defined by instrument end 104. In further embodiments, the user may use an adhesive, such as glue or tape, to couple a supporting device, such as support member 102, to the medical instrument.

In some embodiments, as described above, a user may attach an opposite end of the supporting device, such as patient end 106 of support member 102 described hereinabove, to a surface of a patient's body, for example to a region of the skin of a patient. Using the example of treating pain from the sacroiliac region of a patient, the patient end 106 of support member 102 may be operatively connected to the region of skin on the lower back or buttocks of a patient. As described herein above, the supporting device may be attached to the patient's body in various ways. For example, the user may attach an end of the supporting device to the skin of the patient by, for example, pressing an adhesive portion of the supporting device onto the patient's skin. Alternatively, the user may apply an adhesive to a portion of a surface of the supporting device, and then press the adhesive onto the patient's skin. In yet another embodiment, the user may place a portion of the supporting device on the patient's skin, and may then place tape or other securing means over the supporting device in order to adhere it to the patient's skin. In some embodiments, the means for securing the supporting device to the patient's skin may be removable from the patient's skin without causing unnecessary trauma to the patient.

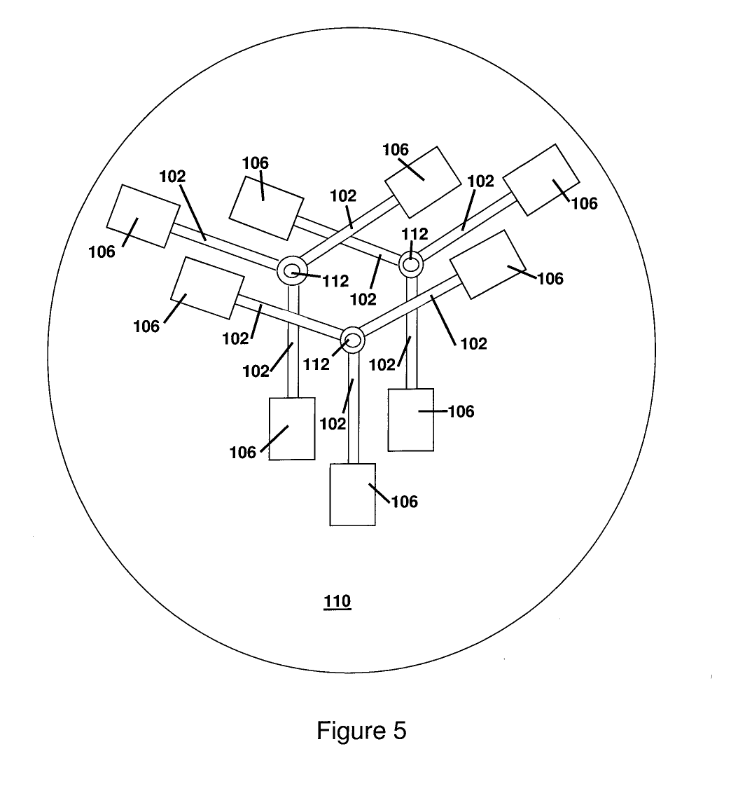

In addition to the above, the user may operatively couple or attach one or more additional support members to the medical instrument and patient in order to further stabilize and/or secure the medical instrument. For example, a user may secure a plurality of support members to the medical instrument in order to achieve the tripod structure shown in FIG. 5. As used herein, the terms “tripod” and “tripodal configuration” generally refer to an object having 3 legs, which may be, for example, support members as described herein. In the embodiment of FIG. 1, one of the tripod legs comprises the medical instrument itself. The additional supporting devices may be connected using the steps described above. The number and position of the additional supporting device(s) may be determined by the user, and may depend on several factors. These factors include, but are not limited to, the location of the medical instrument within the patient, the angle formed between the medical instrument and the patient's skin, and the weight of the medical instrument.

Using embodiments of an apparatus of the present invention, as described herein above, allows a user to independently position one or more of the instrument and patient ends of each individual support member. This flexibility in coupling the supporting apparatus to the medical instrument allows a user to position the individual patient ends of the support members so as to allow for the insertion of a plurality of medical instruments in close proximity to one another.

In one particular such embodiment, the plurality of medical instruments are in contact with a patient's body in close proximity to one another, and the method uses a plurality of support members for stabilizing each of the plurality of medical instruments, each support member comprising an instrument end and a patient end and an elongate support member body extending therebetween, and the support member body is sized to position the patient end at a distance from a point of contact of the medical instrument with the patient's body.

In an embodiment, for each of the medical instruments, the instrument end of each of the plurality of support members are independently coupled to the medical instrument. In addition, for each of the plurality of medical instruments, the patient end of each of the plurality of support members are independently attached to a surface of the patient's body, at a distance from the point of contact, for stabilizing the medical instrument. In some embodiments, the distance at which the patient ends are attached to the patient's body is at least equivalent to the width of a patient end. Attaching the patient end of each of the plurality of support members to a surface of the patient's body at a distance from the point of contact allows for overlapping a support member body associated with one of the plurality of medical instruments with at least a portion of a support member associated with another of the plurality of medical instruments, such that the plurality of medical instruments may be stabilized in close proximity to one another.

Put another way, locating a first patient end of a first support member at a distance from the point of contact of a first medical instrument with the patient's body, where the distance is equivalent to at least the width of the patient end, allows for the insertion and positioning of a second patient end between the point of contact and the first patient end. In such a manner, the effective stabilization region, where no other medical instruments may be positioned, of a first medical instrument being stabilized by support members as described herein, is reduced to the size of the point of contact, since a second medical instrument, also being stabilized by support members as described herein, may be positioned with its point of contact substantially abutting the point of contact of the first medical instrument.

As has been described above with respect to FIG. 1, the user may operatively couple or attach one or more depth stoppers, or other motion limiting members, to the medical instrument in order to prevent one or more of the support member and the medical instrument from moving in a longitudinal direction. In the case of an annular depth stopper, the user may place the one or more depth-stoppers on the medical instrument prior to initiating the procedure. If, however, the depth stopper is somewhat penannular (i.e. an incomplete annulus), for example C-shaped, it may be snapped or pressed onto the medical instrument at any point before or during the procedure. The support member may then be operatively connected to the medical instrument at a point above the depth-stopper, such that the depth stopper may prevent it from sliding down the medical instrument. In some embodiments, the axial position of a depth stopper along the medical instrument is fixed for inserting the medical instrument to a desired depth within the patient's body. In some such embodiments, the support members are fabricated from a substantially rigid material and are supported by a depth stopper located along the medical instrument. In such an embodiment, the user may determine the desired insertion depth of the medical instrument and may position the depth stopper along the medical instrument such that the patient ends of the support members will contact the patient's skin when the desired insertion depth has been reached.

As has been mentioned above, a depth stopper or other motion limiting members may also be positioned on the medical instrument at the surface of the patient's skin in order to provide further stabilization and to limit the depth of insertion of the medical instrument. In some embodiments, one or more depth stoppers may be used without additional support members in order to provide some degree of stabilization to the medical instrument. Additionally, in some embodiments, one or more depth stoppers may be positioned on the medical instrument at the surface of the patient's body and may be used to compress the tissue at the surface of the patient's body in order to help stabilize the medical instrument.

In some embodiments of the present invention, one or more components of the supporting device or apparatus may be positioned after the medical instrument has been itself properly positioned at a desired location on or within a patient's body. For example, in some embodiments, the medical instrument is first inserted into the patient's body and guided to a desired location within the patient's body. At that point, support members are coupled to the medical instrument and attached to the patient's body in order to stabilize the medical instrument. In alternate embodiments, the support members are coupled to the medical instrument prior to inserting the medical instrument into the patient's body but are not attached to the patient's body until the medical instrument has been properly positioned. Such embodiments allow the medical instrument to be re-oriented and repositioned as required prior to attaching the supporting device or apparatus to the patient's body.

Once the procedure has been completed, the user may remove the patient ends of the support member(s) from the patient's skin. This may be accomplished in various ways, depending on how the patient ends are attached to the patient's skin. For example, in some embodiments, the patient ends may be removed by pulling the patient ends off of the skin, or by removing any tape or other securing means that was applied to the skin.

In one particular application, embodiments of the present invention may be used to both stabilize a medical instrument as well to provide an indication as to where the medical instrument should be inserted into the patient's body. For example, it may be desirable to position a plurality of medical instruments at a common distance away from a centre point. In such embodiments, a plurality of patient ends of supporting members of the present invention may be ‘stacked’, or positioned one on top of the other. The supporting members may be coupled to a plurality of medical instruments. If the supporting members are sufficiently rigid, then positioning the patient ends in this way ensures that any medical instruments coupled at the instrument ends of these supporting members are located at a common distance away from the centre point, which is essentially the position of the patient ends. Thus, the supporting members may provide stabilization to the medical instrument and may also assist in positioning the medical instrument appropriately.

Thus, as described herein, embodiments of the present invention provide devices, kits, apparatuses, and methods of use thereof, useful for stabilizing medical instruments contacting a patient's body. Such devices may, in some embodiments, be manufactured at a relatively low cost and may be operable to be selectively coupled and/or removed from the medical instrument. More particularly, embodiments of the present invention may be coupled to the medical instrument and/or attached to the patient's body after the medical instrument has been properly positioned at or within the patient's body. Furthermore, as described herein above, a plurality of medical instruments may be positioned substantially near one another while using embodiments of apparatuses of the present invention to stabilize each of the medical instruments. In addition, in applications involving the insertion of medical instruments into the patient's body, embodiments of apparatuses of the present invention described herein do not limit accessibility to the site at which the instruments are inserted.

The embodiments of the invention described above are intended to be exemplary only. Although one specific application of the method of the present invention has been described, this invention may be practiced in conjunction with various procedures at various sites on or within a patient's body. In addition, it should be appreciated that variations of the disclosed apparatus embodiments are also contemplated. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations may exist. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method for stabilizing a substantially rigid medical instrument, the medical instrument contacting a patient's body at a point of contact, the method using a plurality of support members for stabilizing the medical instrument, each support member comprising an instrument end and a patient end and an elongate support member body extending therebetween, the method comprising:
independently coupling the instrument end of each of the plurality of support members to the medical instrument; and
independently attaching the patient end of each of the plurality of support members to a surface of the patient's body for stabilizing the medical instrument.

2. The method of claim 1, wherein the instrument end of each of the plurality of support members is removably coupled to the medical instrument and selectively movably affixed thereto by frictional engagement.

3. The method of claim 1, wherein the medical instrument is positioned at a desired location prior to attaching the patient ends, of the plurality of support members coupled to the medical instrument, to the patient's body.

4. The method of claim 1, wherein the plurality of support members are coupled to the medical instrument and attached to the patient's body in a tripodal configuration.

5. The method of claim 1, wherein the plurality of support members are coupled to the medical instrument and attached to the patient's body for maintaining a position of the one or more medical instruments during the course of a treatment procedure.

6. The method of claim 5, wherein the treatment procedure comprises a neural ablation procedure.

7. The method of claim 6, wherein the treatment procedure is performed in a sacroiliac region of the patient's body.

8. The method of claim 1, wherein the medical instrument is selected from the group consisting of a probe and a cannula.

9. The method of claim 1, further comprising coupling one or more motion limiting members to the medical instrument for limiting longitudinal motion of one or more of the medical instrument and the plurality of support members coupled to the medical instrument.

10. The method of claim 9, wherein at least one of the one or more motion limiting members is positioned at the surface of the patient's body for compressing tissue at the surface of the patient's body in order to further stabilize the medical instrument.

11. A kit of parts for stabilizing a substantially rigid elongate medical instrument contacting a patient's body at a point of contact, the kit comprising:
at least one instrument support member comprising
an instrument end for removably coupling to the medical instrument, the instrument end at least partially defining an aperture sized for selectively movably affixing the instrument end to the medical instrument by frictional engagement,
an elongate support member body extending from the instrument end for stabilizing the instrument end, and
a patient end extending from the elongate support member body for removably attaching to the patient's body for anchoring the support member body; and
at least one motion limiting member for limiting longitudinal motion of one or more of the medical instrument and the instrument end of the support member.

12. The kit of claim 11, wherein the support member body is sized such that the instrument end of the support member is operable to be coupled to the medical instrument at a distance from the point of contact for substantially limiting angular motion of the medical instrument about the point of contact.

13. The kit of claim 11, wherein the support member body is sized such that the patient end of the support member is operable to be attached to the patient's body at a distance from the point of contact for allowing a plurality of medical instruments to be stabilized in close proximity to one another.

14. The kit of claim 11, wherein the at least one motion limiting member comprises a substantially annular component sized to at least partially circumscribe a portion of the medical instrument for frictionally engaging the medical instrument.

15. The kit of claim 14, comprising a plurality of substantially annular components for frictionally engaging the medical instrument, wherein at least one of the plurality of substantially annular components is operable to be positioned along the medical instrument substantially adjacent the point of contact for limiting longitudinal motion of the medical instrument and wherein at least one other of the plurality of substantially annular components is operable to be positioned substantially adjacent the instrument end of the support member for limiting longitudinal motion of the instrument end of the support member along the medical instrument.

16. The kit of claim 14, wherein the motion limiting member further comprises a treatment composition.

17. The kit of claim 14, wherein the motion limiting member is substantially radiopaque.

18. The kit of claim 14, wherein one or more of the support member and the motion limiting member are color-coded based on a size thereof.

19. The kit of claim 11, wherein a length of the support member body is variable.

20. The kit of claim 11, wherein the support member body is substantially flexible.

21. A device for stabilizing a substantially rigid medical instrument contacting a patient's body at a point of contact, the device comprising:
an instrument end for removably coupling to the medical instrument, the instrument end at least partially defining an aperture sized for selectively movably affixing the instrument end to the medical instrument by frictional engagement;
a plurality of elongate support member bodies extending from the instrument end for stabilizing the instrument end; and
a patient end extending from each of the plurality of elongate support member bodies for removably attaching to the patient's body for anchoring the plurality of support member bodies.
